(19) United States
(12) Patent Application Publication
(10) Pub. No.: US 2008/0108805 A1
(43) Pub. Date: May 8, 2008

(54) Method For The Hybridisation Of Nucleic Acids

(76) Inventors: Uwe Claussen, Jena (DE); Karl-Jurgen Halbhuber, Isserstedt (DE); Thomas Liehr, Jena (DE); Anja Weise, Bad Klosterlausnitz (DE)

Correspondence Address: JORDAN AND HAMBURG LLP, 122 EAST 42ND STREET, SUITE 4000, NEW YORK, NY, 10168, US

(21) Appl. No.: 11/795,996

(22) PCT Filed: Jan. 24, 2006
(86) PCT No.: PCT/DE06/00113

§ 371 Date: Aug. 29, 2007

(30) Foreign Application Priority Data
Jan. 24, 2005 (DE) 10 2005 003 638.4

(51) Int. Cl. C07H 1/00

(52) U.S. Cl. 536/25.3

(57) ABSTRACT

The invention relates to the improvement of in-situ hybridization with nucleic acids and to the shortening of the hybridization method. According to this invention, the method for the hybridization of nucleic acids is carried out under the influence of microwaves at a hybridization temperature of up to a maximum of 37° C.

Said invention is used, for example, for marking and evaluating nucleic acids probes.

### BACKGROUND OF THE INVENTION

The invention relates to a method for the rapid in-situ-hybridization with nucleic acids, for example for marking nucleic acids probes that are to be evaluated.

Fields of application include both in-situ-hybridizations that are to performed under time pressure, e.g. for prenatal diagnoses, and nucleic acids probes that are small or difficult to hybridize and hybridize more reliable by this method.

In-situ hybridizations with nucleic acids are quite known (e.g. Pinkel D, Straume T, Gray J W: Cytogenetic analysis using quantitative, high-sensitivity, fluorescence hybridization, Proc Natl Acad Sci USA 83, 1986, 2934-2938). They are carried out at defined temperatures (mostly 37° C.) and are designed to bond nucleic acids probes (e.g. DNA probes or sondes), which are mostly marked in a special manner (e.g. by fluorescent dyes that illuminate under the fluorescence microscope after the appropriate excitation) on target sequences with the corresponding base homology. Said target sequences can be located in different forms. They can be for example DNA sequences in nuclei, in chromosomes or generally in tissue or sections of tissue, but they can also be already bond to special surfaces, such as glass in combination with array technologies (overview e.g. Forster T, Roy D, Ghazal P: Experiments using microarray technology: limitations and standard operating procedures, J Endocrinol. 178, 2003, 195-204).

It is known that the probe sequences have often difficulties to reach the target sequences (for example, DNA sequences in chromosomal structures, Saitoh Y, Laemmli UK: Metaphase chromosome structure: bands arise from a differential folding path of the highly AT-rich scaffold, Cell 76, 1994, 609-622).

The target sequences can be melted open (denaturing the DNA, for example) particularly by heat before hybridization. (Overview in “FISH Technologies”, Rautenstrauss and Liehr, 1^{st} edition, Springer 2001).

Generally, it must be mentioned that in-situ hybridizations with nucleic acids require relatively much time and need several hours for the reaction. For these reasons, they last till the night or the next day and thus affect the further treatment of the hybridized preparations. Normally, a continuous further processing is not or only seldom possible due to the long reaction times.

Up to now, microwaves have not been used for improving the hybridization efficiency on chromosomal DNA. Neither is such a specific use of microwaves known among experts.

Till now, microwaves have only been used for the improvement of hybridization on tissue sections in the field of immunohistochemistry. (e.g. Coates P J, Hall P A, Butler M G, D'Ardenne A J: Rapid technique of DNA-DNA in situ hybridisation on formalin fixed tissue sections using microwave irradiation, J Clin Pathol 40, 1987, 865-869: Bull J H, Harnden P: Efficient nuclear FISH on paraffin-embedded tissue sections using microwave pretreatment, Biotechniques. 26, 1999, 416-418, 422; Kobayashi K, Kitayama Y, Igarashi H, Yoshino G, Kobayashi T, Kazui T, Sugimura H: Intratumor heterogeneity of centromere numerical abnormality in multiple primary gastric cancers: application of fluorescence in situ hybridization with intermittent microwave irradiation on paraffin-embedded tissue, Jpn J Cancer Res 91, 2000, 1134-1141).

Moreover, microwaves have only been used on chromosomal DNA and tissue sections before the hybridization process to transfer the target sequences (using a thermal effect) from the double-strand form to the single-strand from (melting the double-strand open) and thus to make them ready for a hybridization with probe DNA only now (e.g. Ko E, Rademaker A, Martin R: Microwave decondensation and codenaturation: a new methodology to maximize FISH data from donors with very low concentrations of sperm, Cytogenet Cell Genet 95, 2001, 143-145). This effect has an influence on the usability of the target sequences for hybridization, but not on the efficiency of the subsequent hybridization. However, the experts show a great interest in the reduction of the hybridization times because standard fluorescent in-situ protocols contain six steps in the main. Each of these steps is important to achieve a hybridization result (practical details: Levy E R, Herrington C S: Non-isotopic methods in molecular biology: a practical approach, 1995 Oxford University Press; Choo KHA: Methods in Molecular Biology, Vol. 33: In situ hybridisation Protocols, 1994, Humana Press Inc.; Totowa N.J., Wilkinson D G: In situ hybridization: a practical approach. 1992, Oxford University Press): 1^{st} preparation of the target sequence (fixation and predigestion), 2^{nd} probe preparation and probe marking, 3^{rd} denaturation of the target sequence and probe, 4^{th} hybridization of the probe on the target sequence, 5^{th} posthybridization washings, and 6^{th} detection.

The most time-consuming step is by far step 4. Depending on the probe type, it lasts two to four hours (centrometer probes) or one or several days (single copy and very small probes).

### SUMMARY OF THE INVENTION

Therefore, the task of the invention is to improve in-situ hybridization with nucleic acids and to shorten the hybridization process.

### BRIEF DESCRIPTION OF THE INVENTION

According to this invention, the method for the hybridization of nucleic acids is carried out under the influence of microwaves.

Surprisingly, it turned out that the hybridization under the influence of microwaves requires considerably less time than the conventional method. The mentioned and otherwise normally long time required for the hybridization can be considerably reduced thanks to this invention so that the further treatment of the preparations after the hybridization (e.g. the evaluation under the microscope) is not problematic with regard to the time factor and can be carried out on the same day (and thus directly after the hybridization). Mostly, this has been very difficult till now. Moreover, in the result of the recommended microwave treatment the hybridization results give a considerably better quality impression that becomes obvious in more specific hybridization signals. This fact is particularly decisive in the use of small and single copy nucleic acids probes the conventional hybridization of which often leads to results with rich backgrounds that cannot be evaluated.

For the hybridization process itself, the inventive microwave effect is mainly independent of the heat development at the target sequences. It is rather to assume a microwave excitation of the molecule motions that, in a steric manner, makes it easier for the probe sequences to reach the target sequences and even improves this process.

The nucleic acids hybridization under the influence of microwaves is preferentially carried out at a hybridization temperature of up to a maximum of 37° C. Advantageously, the hybridization temperature can be controlled by means of a cold water bath and by defining the time of the microwave influence according to the water temperature and water volume of said cold water bath by using calibration curves in such a way that a water temperature of a maximum of 37° C. is reached at the end of the microwave effect time.

In the following, one example (instruction for the production of preparations of human metaphase chromosomes from lymphocytes for marking purposes and for the fluorescence microscopic analysis) explains the invention in detail.

Instruction:

Produce preparations of human metaphase chromosomes from lymphocytes in a conventional method and denature the chromosomal DNA in a conventional process, too (by applying heat).

Select a DNA probe (e.g. a human BAC clone) marked by a fluorescent dye and denature its DNA in a conventional process (e.g. by means of heat).

Add the denatured probe DNA over the denatured target DNA (e.g. on the slide) and transfer both for hybridization in to a small water bath at 37° C.

Put the water bath into a microwave oven and perform the microwave treatment in form of short microwave impulses at temporal intervals (for example, four to six times within half an hour at 600 W during one minute).

Afterwards, prepare the slide for the fluorescence microscopic analysis.

1. Method for the hybridization of nucleic acids, comprising treating nucleic acids with microwaves thereby to hybridize the nucleic acids.

2. Method according to claim 1, wherein the hybridization of nucleic acids is performed at a temperature of up to a maximum of 37° C.

3. Method according to claim 2, wherein the nucleic acid to be hybridized is introduced into a water bath having a temperature below 37° C. and the water bath is heated by the microwaves for a period of time until the water bath reaches a temperature of 37° C., the period of time being predetermined based on the initial temperature and volume of the water bath.

4. Method according to in claim 1, wherein the microwaves are applied as short microwave impulses.

5. Method according to claim 4, wherein the treatment consists of four to six microwave impulses within half an hour.
